(19) United States
(12) Patent Application Publication
(10) Pub. No.: US 2025/0087298 A1
(43) Pub. Date: Mar. 13, 2025

(54) PROTEIN SEQUENCE BASED VIRTUAL SCREENING METHOD AND DEVICE, EQUIPMENT AND STORAGE MEDIUM

(71) Applicant: Ainnocence INC, MIAMI, FL (US)

(72) Inventors: Yutong Jin, Zhengzhou (CN); Junfeng Wu, Shanghai (CN); Lurong Pan, Vestavia Hill, AL (US)

(21) Appl. No.: 18/454,092

(22) Filed: Sep. 8, 2023

(51) Int. Cl. G16B 15/30; G16B 5/00

(52) U.S. Cl. CPC G16B 15/30; G16B 5/00

(57) ABSTRACT

A protein virtual screening method and device, equipment, and a storage medium, falling within the field of drug discovery. The method comprises: acquiring a training sample set, wherein the set comprises source and sample data corresponding to the source data; performing unsupervised pre-training on a Transformer model with the source data as input and the sample data as verification, and generating a one-dimensional or multi-dimensional symmetric matrix for the protein sequence and the ligand sequence; coupling two matrices for the protein sequence and the ligand sequence into a multi-dimensional symmetric matrix, and using the matrix as an input of an hidden layer of a BILSTM network model; fitting the experimental measurement classification and regression value of protein and small molecule interaction by the Bilstm network model to obtain to a trained screening model; and predicting different protein prediction tasks by the screening model to output prediction results.

### TECHNICAL FIELD OF THE INVENTION

The present invention relates to the field of drug discovery, and in particular to a protein virtual screening method and device, equipment and a storage medium.

### BACKGROUND OF THE INVENTION

Drug discovery has been a time and money consuming process for a long time. With the development of computer technology, computational methods are widely used in drug research and development. Virtual drug screening is one of the most valuable technologies. Among them, according to the virtual screening artificial intelligence method based on the protein crystal structure, the three-dimensional structure information of protein targets is used to predict the binding of protein to drug small molecules.

A molecular docking method is often used in the common virtual screening method based on the protein crystal structure ([M. Drwal and R. Griffith. Combination of ligand- and structure-based methods in virtual screening], [S. Ghosh, A. Nie, J. An, and Z. Huang. Structure-based virtual screening of chemical libraries for drug discovery.], [J. Gilmer, S. Schoenholz, P. Riley, O. Vinyals, and G. Dahl. Neural message passing for quantum chemistry.]). According to the method, molecular dynamics and quantum chemistry methods are used to simulate the binding process of small molecules to proteins, involving the search of binding sites and using heuristic functions to calculate the spatial position of small molecules, which is computationally intensive, and causes many possible results with some uncertainty. Molecular docking-based methods, on the other hand, is required to rely on information on the three-dimensional structure of proteins, and the vast majority of biological proteins are not known, thus limiting their widespread use in the pharmaceutical industry.

Therefore, it is necessary to establish a set of artificial intelligence technology that can surpass the protein sequence-based virtual screening to expand the range of artificial intelligence training data and improve the accuracy and speed of virtual screening.

### SUMMARY OF THE INVENTION

It Therefore, in order to overcome the above-mentioned disadvantages of the prior art, the present invention provides a protein virtual screening method and device, equipment and a storage medium for efficiently, quickly and accurately predicting an interaction relationship between a protein and a drug small molecule based on a protein sequence.

In order to achieve the above object, the present invention provides a protein virtual screening method comprising: acquiring a training sample set, wherein the training sample set comprises source data and sample data corresponding to the source data; the source data comprises protein sequences and ligand sequences of a small molecule compound which can bind to the protein; the sample data comprises subsequences of binding sites of the protein and the small molecule compound; performing unsupervised pre-training on a Transformer model with the source data as input and the sample data as verification, and respectively generating a one-dimensional or multi-dimensional symmetric matrix for the protein sequence and the ligand sequence; coupling two matrices for the protein sequence and the ligand sequence into a multi-dimensional symmetric matrix, and using the multi-dimensional symmetric matrix as an input of an hidden layer of a BILSTM network model; fitting the experimental measurement classification and regression value of protein and small molecule interaction by the Bilstm network model to obtain to a trained screening model; and predicting different protein prediction tasks by the screening model to output prediction results.

In an embodiment, the acquiring a training sample set comprises: executing a masking strategy based on a BERT-type masking language model, randomly masking binding sites of protein targets, obtaining a discontinuous protein sequence characterization, and using the discontinuous protein sequence characterization as sample data; and correspondingly storing the source data and the sample data to obtain training sample sets.

In an embodiment, the performing unsupervised pre-training on a Transformer model with the source data as input and the sample data as verification, and respectively generating a one-dimensional or multi-dimensional symmetric matrix for the protein sequence and the ligand sequence comprises: inputting the training sample sets of the protein sequence and the ligand sequence into an input embedding layer of the Transformer model, respectively; embedding the training sample sets by the input embedding layer of the Transformer model; inputting the embedded training sample sets into an intermediate hidden layer of the Transformer model; learning feature representation of the training sample sets by the intermediate hidden layer of the Transformer model; and outputting the learned feature representation by an output prediction layer of the Transformer model, the feature representation being a one-dimensional or multi-dimensional symmetric matrix.

In an embodiment, the coupling two matrices for the protein sequence and the ligand sequence into a multi-dimensional symmetric matrix comprises: coupling two matrices for the protein sequence and the ligand sequence by an QSAR model to obtain a multi-dimensional symmetric matrix.

In an embodiment, the fitting the experimental measurement classification and regression value of protein and small molecule interaction by the Bilstm network model to obtain to a trained screening model comprises: acquiring a protein sequence X=(x1, . . . , xL), i=1, . . . , L, wherein X is all amino acids; represents a point mutation at a position I, a mutation sequence x ( )=(x1, . . . , xi−1, xi+1, . . . , xL), and a sequence context x[L]\{i}=(x1, . . . , xi−1, xi+1, . . . , xL); encoding the sequence context by a vector, wherein fe is an embedding function mapping a discrete sequence to a D-dimensional continuous space; the embedding function is instantiated by a bidirectional LSTM neural network and is connected to the output of a final LSTM layer to form an embedding vector so as to obtain, wherein gf is the output of first several layers, the first several layers are in a forward input, and LSTMf is a last layer of the forward LSTM; gr and gf are defined similarly and in opposite directions; LSTMr and LSTMf are defined similarly and in opposite directions; fitting the experimental measurement classification and regression value of protein and small molecule interaction by embedding vector zi by means of the learning transformation and softmax function the to obtain to a trained screening model; the softmax function is, where W and b are learning parameters.

A protein virtual screening device comprises: a sample set processing module configured for acquiring a training sample set, wherein the training sample set comprises source data and sample data corresponding to the source data; the source data comprises protein sequences and ligand sequences of a small molecule compound which can bind to the protein; the sample data comprises subsequences of binding sites of the protein and the small molecule compound; an unsupervised pre-training module configured for performing unsupervised pre-training on a Transformer model with the source data as input and the sample data as verification, and respectively generating a one-dimensional or multi-dimensional symmetric matrix for the protein sequence and the ligand sequence; an input setting module configured for coupling two matrices for the protein sequence and the ligand sequence into a multi-dimensional symmetric matrix, and using the multi-dimensional symmetric matrix as an input of an hidden layer of a BILSTM network model; a model training module configured for fitting the experimental measurement classification and regression value of protein and small molecule interaction by the Bilstm network model to obtain to a trained screening model; and a prediction module configured for predicting different protein prediction tasks by the screening model to output prediction results.

A computer equipment comprising a memory and a processor, with the memory having stored thereon a computer program, is characterized in that the processor, when executing the computer program, implements the steps of the method described above.

A computer-readable storage medium having stored thereon a computer program is characterized in that the computer program, when being executed by a processor, carries out the steps of the method as described above.

The advantages of the present invention over the prior art are as follows: the direct prediction of the interaction between proteins and small molecules based on the sequence information of protein greatly expands the application of virtual screening technology in drug design of different biological targets, especially those with no three-dimensional structure information. Furthermore, by using large data and deep learning methods, the computational cost can be saved more than 10000 times compared with molecular dynamics, quantum mechanics and quantum chemistry, and the computational speed is greatly improved. In addition, the elastogram neural network simulation can better adapt to the simulation of highly flexible protein, thus reducing the flexibility gap between the protein conformation simulation and the real physiological structure protein conformation, thus reducing the simulation error and significantly improving the accuracy of protein and small molecule binding prediction. The present application can also establish a standard virtual screening method flow based on protein sequences, which plays a fundamental role in the development of such methods in the future.

### BRIEF DESCRIPTION OF DRAWINGS

The In order to explain the embodiments of the present disclosure or the technical solutions in the prior art more clearly, the following will briefly introduce the drawings that need to be used in the description of the embodiments or the prior art. Obviously, the drawings in the following description are only some embodiments of the present disclosure. For those of ordinary skill in the art, other drawings can be obtained based on these drawings without creative work.

FIG. 1 is a schematic flow diagram of a protein virtual screening method according to an embodiment of the present invention;

FIG. 2 is a schematic flow diagram of a protein virtual screening step according to an embodiment of the present invention;

FIG. 3 is a structure block diagram of a protein virtual screening device according to an embodiment of the present invention; and

FIG. 4 is an internal structure block diagram of computer equipment according to an embodiment of the invention.

### DETAILED DESCRIPTION

The embodiments of this disclosure are described in detail in combination with the accompanying drawings below.

Additional advantages and utility of the present disclosure will become readily apparent to those skilled in the art from the following detailed description, taken in conjunction with the accompanying drawings, illustrating by way of example embodiments of the present disclosure. Obviously, the described embodiments are some, but not all, embodiments of the present disclosure. The present disclosure may be practiced or applied in various other specific embodiments, and the details of the description may be modified or varied from one viewpoint or application without departing from the spirit of the disclosure. It should be noted that the embodiments and the features in the embodiments below may be combined with one another without conflict. Based on the embodiments in the disclosure, all other embodiments obtained by a person skilled in the art without involving any inventive effort are within the scope of protection of the disclosure.

It is intended that the following describe various aspects of embodiments within the scope of the appended claims. It should be apparent that aspects described herein may be embodied in a wide variety of forms and that any specific structure and/or function described herein is merely illustrative. Based on the present disclosure, one skilled in the art will appreciate that one aspect described herein can be implemented independently of any other aspects, and that two or more of these aspects can be combined in various ways. For example, the equipment may be implemented and/or a method may be practiced using any number of the aspects set forth herein. Additionally, such an equipment can be implemented and/or such a method can be practiced using other structure and/or functionality in addition to one or more of the aspects set forth herein.

It should be noted that the figures provided in the following examples merely illustrate the basic idea of the present disclosure in a schematic way. Thus, only the components related to the present disclosure are shown in the drawings instead of being drawn according to the number, shape and size of the components in an actual implementation. In an actual implementation, the type, number and proportion of the components may be changed at will, and the layout of the components may be more complicated.

In addition, in the following description, specific details are provided to facilitate a thorough understanding of the examples. However, it will be understood by those skilled in the art that the aspects may be practiced without these specific details.

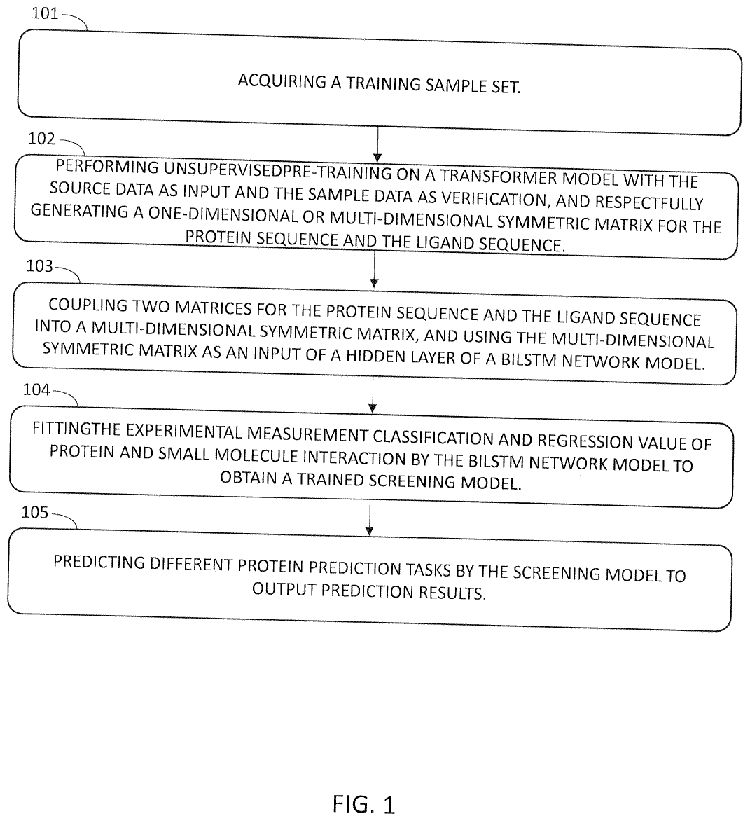

As shown in FIG. 1, the embodiments of the present disclosure provide a protein virtual screening method, which can be applied on a terminal or a server. The terminal can be, but is not limited to, various personal computers, notebook computers, smart phones, tablet computers and portable intelligent devices. The server can be realized by an independent server or a server cluster composed of a plurality of servers. The method includes the following steps.

Step 101, a training sample set is acquired, wherein the training sample set includes source data and sample data corresponding to the source data; the source data includes protein sequences and ligand sequences of a small molecule compound which can bind to the protein; the sample data includes subsequences of binding sites of the protein and the small molecule compound.

The source data includes the sequence of the protein, and the sequence of the ligand of the small molecule compound to which the protein can bind. The sample data includes subsequences of binding sites for proteins and small molecule compounds. The server may obtain a training sample set directly from the database or perform secondary processing on the training sample set. Small molecule compounds have an experimental record of enzyme activity in proteins within the protein data bank (pdb).

Step 102, unsupervised pre-training is performed on a Transformer model with the source data as input and the sample data as verification, and a one-dimensional or multi-dimensional symmetric matrix is respectively generated for the protein sequence and the ligand sequence.

The server performs unsupervised pre-training on a Transformer model with the source data as input and the sample data as verification, and respectively generates a one-dimensional or multi-dimensional symmetric matrix for the protein sequence and the ligand sequence. Based on the Transformer model architecture, the server can use a bulk of unlabeled source data and a BERT-type Masked Language Model to perform the unsupervised pre-training, and finally get a powerful protein pre-training model. The final output of this model is a one-dimensional or multi-dimensional symmetric matrix. The Transformer model includes an input embedding layer, an intermediate hiding layer and an output prediction layer which are connected in sequence. The intermediate hiding layer is composed of N Transformer modules, and each Transformer module includes a multi-head attention layer, a first Dropout layer, a first Add & Norm layer, a feed-forward layer, a second Dropout layer and a second Add & Norm layer which are connected in sequence. Add represents a residual connection for preventing network degradation. Norm denotes Layer Normalization, which is used to normalize the activation values of each layer. Each Transformer module includes two sub-layers, a Multi-head Attention layer and a Feed-forward layer. Each sub-layer is followed by Dropout, Add and Norm operations, so as to learn a feature representation of masking a binding site for a protein and a small molecule sequence. After passing through all the Transformer modules, the Transformer model has fully learned the feature representation of masking a binding site. Finally, the learned feature representation is input to the output prediction layer to predict a binding site at a masking position.

Step 103, two matrices are coupled for the protein sequence and the ligand sequence into a multi-dimensional symmetric matrix, and the multi-dimensional symmetric matrix is used as an input of an hidden layer of a BiLSTM network model.

The server couples the two matrices for the protein sequence and the ligand sequence into a multi-dimensional symmetric matrix, and uses the multi-dimensional symmetric matrix as an input of an hidden layer of a BILSTM network model. The server accesses the same input sequence into the two LSTM forward and backward respectively, and then connects the hidden layers of the two networks together to access the output layer together for prediction.

Step 104, the experimental measurement classification and regression value of protein and small molecule interaction are fit by the Bilstm network model to obtain to a trained screening model.

The server fits the experimental measurement classification and regression value of protein and small molecule interaction by the Bilstm network model to obtain to a trained screening model.

Step 105, different protein prediction tasks are predicted by the screening model to output prediction results.

The server predicts different protein prediction tasks by the screening model to output prediction results. The predicted result may be a protein sequence, or the presence or absence of the protein sequence may be output as needed, etc.

According to the above-mentioned method, the direct prediction of the interaction between proteins and small molecules based on the sequence information of protein greatly expands the application of virtual screening technology in drug design of different biological targets, especially those with no three-dimensional structure information. Furthermore, by using large data and deep learning methods, the computational cost can be saved more than 10000 times compared with molecular dynamics, quantum mechanics and quantum chemistry, and the computational speed is greatly improved. In addition, the elastogram neural network simulation can better adapt to the simulation of highly flexible protein, thus reducing the flexibility gap between the protein conformation simulation and the real physiological structure protein conformation, thus reducing the simulation error and significantly improving the accuracy of protein and small molecule binding prediction. The present application can also establish a standard virtual screening method flow based on protein sequences, which plays a fundamental role in the development of such methods in the future.

In an embodiment, the acquiring a training sample set includes the steps of: executing a masking strategy based on a BERT-type masking language model, randomly masking binding sites of protein targets, obtaining a discontinuous protein sequence characterization, and using the discontinuous protein sequence characterization as sample data; and correspondingly storing the source data and the sample data to obtain training sample sets.

The server executes a masking strategy based on a BERT-type masking language model, randomly masks binding sites of protein targets to obtain a discontinuous protein sequence characterization, and uses the discontinuous protein sequence characterization as sample data The server randomly masks the binding sites of the protein targets, and can randomly mask k binding site data of n binding site data. The masking strategies may include that the masked k binding site data accounts for 30-40% of the n binding site data, wherein 80% of the masked k binding site data is directly masked, another 10% of the masked k binding site data is replaced with other proteins, and the remaining 10% of the masked k binding site data remains unchanged.

The server stores the source data and the sample data correspondingly to obtain training sample sets.

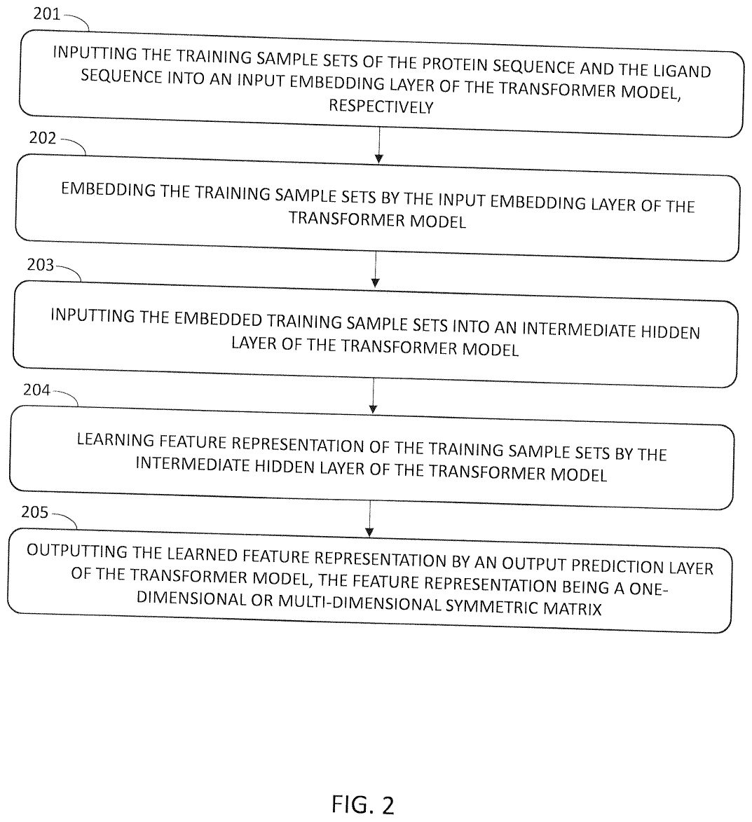

As shown in FIG. 2, in an embodiment, the performing unsupervised pre-training on a Transformer model with the source data as input and the sample data as verification and respectively generating a one-dimensional or multi-dimensional symmetric matrix for the protein sequence and the ligand sequence includes the following steps.

Step 201, the training sample sets of the protein sequence and the ligand sequence are input into an input embedding layer of the Transformer model, respectively.

The server may represent the source data X={x1, x2, . . . , xn} as an embedding matrix[e1, e2, . . . , en], where each column ei in the matrix represents an embedding vector for the corresponding item. The protein sequence and the ligand sequence are different embedded matrices, and thus the protein sequence and the ligand sequence may be one-dimensional or multi-dimensional symmetric matrices. The matrix for the protein sequences is obtained by vector encoding amino acids. For example, an ith amino acid xi on a protein can be composed into a vector eij(xi; xj) by using a single point term representing itself and a coupling term of a paired amino acids xj. The vectors eij(xi; xj) of all amino acids on the protein are combined to obtain a multi-dimensional symmetric matrix for the protein sequences. The ligand sequences can also be composed by using a single point term representing itself and a coupling term of a paired amino acid, resulting in a ligand vector.

Step 202, the training sample sets are embedded by the input embedding layer of the Transformer model.

The server embeds the training sample sets by the input embedding layer of the Transformer model. The server may use dimension reduction techniques to project a matrix of all protein sequences in the training sample set to a low dimension. The dimension reduction technique can be a common data dimension reduction technique, and the redundancy between data can be eliminated by the dimension reduction technique, which can determine the coding structure and stability of the protein, etc.

Step 203, the embedded training sample sets are input into an intermediate hidden layer of the Transformer model.

The server he embedded training sample sets into an intermediate hidden layer of the Transformer model. The Transformer model learns the likelihood that a particular amino acid will appear at a certain position by using all other amino acids surrounding the particular amino acid as context. During the training process, the Transformer model gradually changes its internal dynamics (coded as hidden state vectors) to maximize the prediction accuracy. The server also uses the hidden state vectors of the Transformer model as another protein sequence representation of the prediction model to capture the global protein sequence context, enabling the complement of the local evolution context representation.

Step 204, the feature representation of the training sample sets is learned by the intermediate hidden layer of the Transformer model.

The server learns the feature representation of the training sample sets by the intermediate hidden layer of the Transformer model. The intermediate hidden layer (e.g., a recursive neural network) of the server Transformer model takes these sequence representations as inputs and learns the relationship of the sequences to the functions.

Step 205, the learned feature representation is output by an output prediction layer of the Transformer model, and the feature representation is a one-dimensional or multi-dimensional symmetric matrix.

The server outputs the learned feature representation by an output prediction layer of the Transformer model, the feature representation being a one-dimensional or multi-dimensional symmetric matrix.

In an embodiment, the coupling two matrices for the protein sequence and the ligand sequence into a multi-dimensional symmetric matrix includes: coupling two matrices for the protein sequence and the ligand sequence by an QSAR model to obtain a multi-dimensional symmetric matrix. According to the QSAR model, a mathematical model is used to describe the relationship between a molecular structure and a certain biological activity of a molecule. The basic hypothesis of QSAR is that the molecular structure of a compound contains information determining its physical, chemical and biological properties, and these physical and chemical properties further determine the biological activity of the compound. Furthermore, there should also be some correlation between the molecular structural property data of the compound and its biological activity.

In an embodiment, the fitting the experimental measurement classification and regression value of protein and small molecule interaction by the Bilstm network model to obtain to a trained screening model includes: acquiring a protein sequence X=(x1, . . . , xL), i=1, . . . , L, wherein X is all amino acids; represents a point mutation at a position I, a mutation sequence x ( )=(x1, . . . , xi−1, xi+1, . . . , xL), and a sequence context x[L]\{i}=(x1, . . . , xi−1, xi+1, . . . , xL);
encoding the sequence context by a vector, wherein fe is an embedding function mapping a discrete sequence to a D-dimensional continuous space; the embedding function is instantiated by a bidirectional LSTM neural network and is connected to the output of a final LSTM layer to form an embedding vector so as to obtain, wherein gf is the output of first several layers, the first several layers are in a forward input, and LSTMf is a last layer of the forward LSTM; gr and gf are defined similarly and in opposite directions; LSTMr and LSTMf are defined similarly and in opposite directions;
fitting the experimental measurement classification and regression value of protein and small molecule interaction by embedding vector zi by means of the learning transformation and softmax function the to obtain to a trained screening model;
the softmax function is, where W and b are learning parameters.

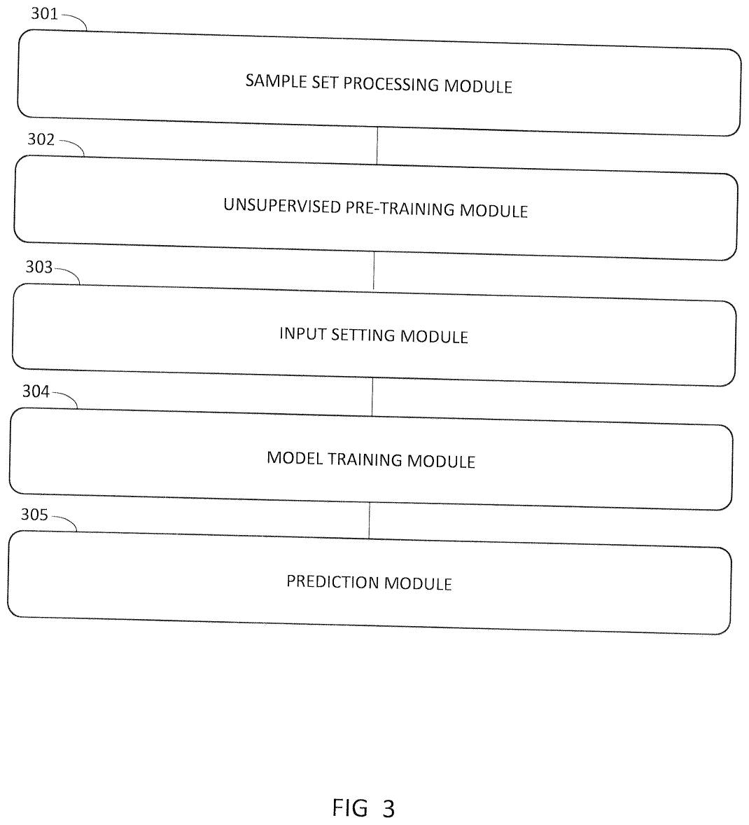

In an embodiment, as shown in FIG. 3, a protein virtual screening device is provided that includes a sample set processing module 301, an unsupervised pre-training module 302, an input setting module 303, a model training module 304, and a prediction module 305.

The sample set processing module 301 is configured for acquiring a training sample set, wherein the training sample set includes source data and sample data corresponding to the source data; the source data includes protein sequences and ligand sequences of a small molecule compound which can bind to the protein; the sample data includes subsequences of binding sites of the protein and the small molecule compound.

The unsupervised pre-training module 302 is configured for performing unsupervised pre-training on a Transformer model with the source data as input and the sample data as verification, and respectively generating a one-dimensional or multi-dimensional symmetric matrix for the protein sequence and the ligand sequence.

The input setting module 303 is configured for coupling two matrices for the protein sequence and the ligand sequence into a multi-dimensional symmetric matrix, and using the multi-dimensional symmetric matrix as an input of an hidden layer of a BILSTM network model.

The model training module 304 is configured for fitting the experimental measurement classification and regression value of protein and small molecule interaction by the Bilstm network model to obtain to a trained screening model.

The prediction module 305 is configured for predicting different protein prediction tasks by the screening model to output prediction results.

In an embodiment, the sample set processing module includes:
a masking unit configured for executing a masking strategy based on a BERT-type masking language model, randomly masking binding sites of protein targets, obtaining a discontinuous protein sequence characterization, and using the discontinuous protein sequence characterization as sample data; and
a storage unit configured for correspondingly storing the source data and the sample data to obtain training sample sets.

In an embodiment, the unsupervised pre-training module includes:
an input unit configured for inputting the training sample sets of the protein sequence and the ligand sequence into an input embedding layer of the Transformer model, respectively;
an embedding unit configured for embedding the training sample sets by the input embedding layer of the Transformer model;
a hiding unit configured for inputting the embedded training sample sets into an intermediate hidden layer of the Transformer model;
a representation unit configured for learning feature representation of the training sample sets by the intermediate hidden layer of the Transformer model; and
an output unit configured for outputting the learned feature representation by an output prediction layer of the Transformer model, the feature representation being a one-dimensional or multi-dimensional symmetric matrix.

In an embodiment, the model training module includes:
a sequence acquisition unit configured for acquiring a protein sequence X=(x1, . . . , xL), i=1, . . . , L, wherein X is all amino acids; represents a point mutation at a position I, a mutation sequence x ( )=(x1, . . . , xi−1, xi+1, . . . , xL), and a sequence context x[L]\{i}=(x1, . . . , xi−1, xi+1, . . . , xL);
an encoding unit configured for encoding the sequence context by a vector, wherein fe is an embedding function mapping a discrete sequence to a D-dimensional continuous space; the embedding function is instantiated by a bidirectional LSTM neural network and is connected to the output of a final LSTM layer to form an embedding vector so as to obtain, wherein gf is the output of first several layers, the first several layers are in a forward input, and LSTMf is a last layer of the forward LSTM; gr and gf are defined similarly and in opposite directions; LSTMr and LSTMf are defined similarly and in opposite directions;
a training unit configured for fitting the experimental measurement classification and regression value of protein and small molecule interaction by embedding vector zi by means of the learning transformation and softmax function the to obtain to a trained screening model;
the softmax function is, where W and b are learning parameters.

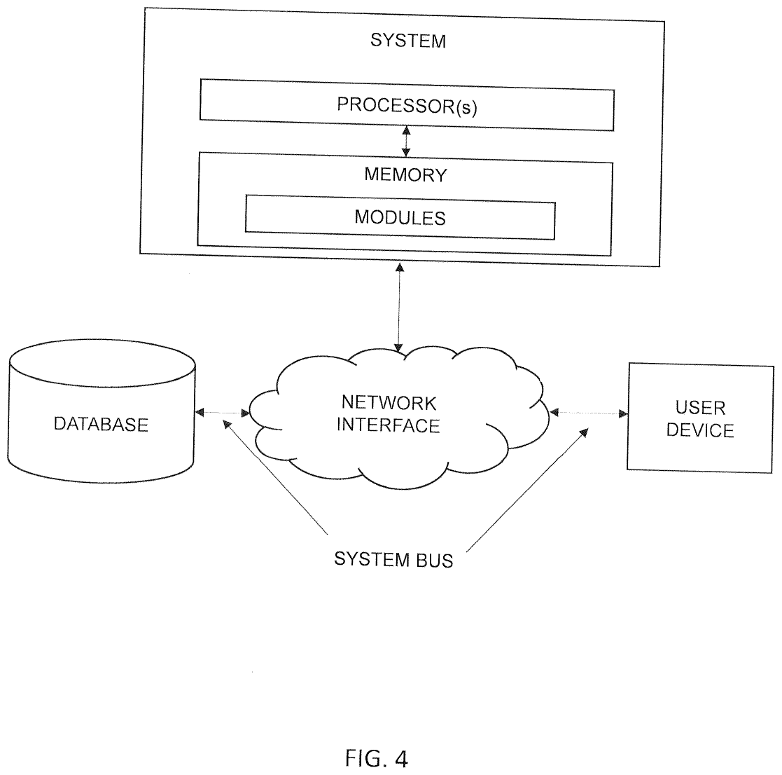

In an embodiment, a computer device is provided, which may be a server, the internal structure of which may be as shown in FIG. 4. The computer device includes a processor, a memory, a network interface, and a database connected via a system bus. The processor of the computer device is configured to provide computing and control capabilities. The memory of the computer device includes a non-volatile storage medium and an internal memory. The non-volatile storage medium stores an operating system, computer programs, and a database. The internal memory provides an environment for the operating system and computer programs in the non-volatile storage medium. The computer equipment's database is used to store protein sequences, training sample sets, etc. The network interface of the computer device is adapted to communicate with an external terminal via a network connection. The computer program when executed by a processor implements a protein virtual screening method.

It will be appreciated by those skilled in the art that the structure shown in FIG. 4 is merely a block diagram of a portion of the structure relevant to the solution of the present application and does not constitute a limitation on the computer equipment to which the solution of the present application is applied. Particularly, the computer equipment may include more or less components than those shown in the drawings, or some components that may be combined, or have a different arrangement of components.

A computer equipment includes a memory and one or more processors, with the memory having stored therein a computer program which when executed by the processors performs the steps of the protein virtual screening method as provided in any of the embodiments of the present application.

One or more computer-readable storage media are provided. The computer programs, when executed by one or more processors, cause the one or more processors to perform the steps of the protein virtual screening method provided in any one of the embodiments of the present application.

While the disclosure has been described with reference to particular embodiments thereof, the scope of this disclosure is not limited thereto. Various changes or equivalents within the technique scope of the disclosure may occur to those skilled in the art without departing from the scope of the disclosure. Accordingly, the scope of protection of this disclosure is subject to the scope of the claims.

1. A protein virtual screening method, characterized by comprising:
acquiring a training sample set, wherein the training sample set comprises source data and sample data corresponding to the source data; the source data comprises protein sequences and ligand sequences of a small molecule compound which can bind to the protein; the sample data comprises subsequences of binding sites of the protein and the small molecule compound;
performing unsupervised pre-training on a Transformer model with the source data as input and the sample data as verification, and respectively generating a one-dimensional or multi-dimensional symmetric matrix for the protein sequence and the ligand sequence;
coupling two matrices for the protein sequence and the ligand sequence into a multi-dimensional symmetric matrix, and using the multi-dimensional symmetric matrix as an input of an hidden layer of a BILSTM network model;
fitting the experimental measurement classification and regression value of protein and small molecule interaction by the Bilstm network model to obtain to a trained screening model; and
predicting different protein prediction tasks by the screening model to output prediction results.

2. The method according to claim 1, characterized in that the acquiring a training sample set comprises:
executing a masking strategy based on a BERT-type masking language model, randomly masking binding sites of protein targets, obtaining a discontinuous protein sequence characterization, and using the discontinuous protein sequence characterization as sample data; and
correspondingly storing the source data and the sample data to obtain training sample sets.

3. The method according to claim 1, characterized in that the performing unsupervised pre-training on a Transformer model with the source data as input and the sample data as verification, and respectively generating a one-dimensional or multi-dimensional symmetric matrix for the protein sequence and the ligand sequence comprises:
inputting the training sample sets of the protein sequence and the ligand sequence into an input embedding layer of the Transformer model, respectively;
embedding the training sample sets by the input embedding layer of the Transformer model;
inputting the embedded training sample sets into an intermediate hidden layer of the Transformer model;
learning feature representation of the training sample sets by the intermediate hidden layer of the Transformer model; and
outputting the learned feature representation by an output prediction layer of the Transformer model, the feature representation being a one-dimensional or multi-dimensional symmetric matrix.

4. The method according to claim 1, characterized in that the coupling two matrices for the protein sequence and the ligand sequence into a multi-dimensional symmetric matrix comprises:
coupling two matrices for the protein sequence and the ligand sequence by an QSAR model to obtain a multi-dimensional symmetric matrix.

5. The method according to claim 1, characterized in that the fitting the experimental measurement classification and regression value of protein and small molecule interaction by the Bilstm network model to obtain to a trained screening model comprises:
acquiring a protein sequence X=(x₁, . . . , X_{L}), i=1, . . . , L, wherein X is all amino acids; \tilde{x}ᵢ represents a point mutation at a position I, a mutation sequence x(\tilde{x}ᵢ)=(x₁, . . . , xᵢ₋₁, xᵢ₊₁, . . . , x_{L}), and a \tilde{x}ᵢ sequence context x_{[L]\{i}}=(x₁, . . . , x₋₁, xᵢ₊₁, . . . , x_{L});
encoding the sequence context by a vector zᵢ=fₑ(x_{[L]\{i}}), wherein fₑ is an embedding function X^{L-1}> [symbol] ^{D} mapping a discrete sequence to a D-dimensional continuous space; the embedding function is instantiated by a bidirectional LSTM neural network and is connected to the output of a final LSTM layer to form an embedding vector so as to obtain
$${z}_{i} = [ {LSTM}_{f} ( {g}_{f} ( {x}_{1} , … , {x}_{i - 1} ) ) ; {LSTM}_{r} ( {g}_{r} ( {x}_{i + 1} , … , {x}_{L} ) ) ]$$
wherein g_{f} is the output of first several layers, the first several layers are in a forward input, and LSTM_{f} is a last layer of the forward LSTM; gᵣ and g_{f} are defined similarly and in opposite directions; LSTMᵣ and LSTM_{f} are defined similarly and in opposite directions; and
fitting the experimental measurement classification and regression value of protein and small molecule interaction by embedding vector zi by means of the learning transformation and softmax function the to obtain to a trained screening model;
the softmax function is
p(xᵢ|x_{[L]\{i}})=p(xᵢ|zᵢ)=softmax(Wzᵢ+b),
where W and b are learning parameters.

6. A protein virtual screening device, characterized in that the device comprises:
a sample set processing module configured for acquiring a training sample set, wherein the training sample set comprises source data and sample data corresponding to the source data; the source data comprises protein sequences and ligand sequences of a small molecule compound which can bind to the protein; the sample data comprises subsequences of binding sites of the protein and the small molecule compound;
an unsupervised pre-training module configured for performing unsupervised pre-training on a Transformer model with the source data as input and the sample data as verification, and respectively generating a one-dimensional or multi-dimensional symmetric matrix for the protein sequence and the ligand sequence;
an input setting module configured for coupling two matrices for the protein sequence and the ligand sequence into a multi-dimensional symmetric matrix, and using the multi-dimensional symmetric matrix as an input of an hidden layer of a BiLSTM network model;
a model training module configured for fitting the experimental measurement classification and regression value of protein and small molecule interaction by the Bilstm network model to obtain to a trained screening model; and
a prediction module configured for predicting different protein prediction tasks by the screening model to output prediction results.

7. A computer equipment comprising a memory and a processor, with the memory having stored thereon a computer program, characterized in that the processor, when executing the computer program, implements the steps of the method of claim 1.

8. A computer-readable storage medium having stored thereon a computer program, characterized in that the computer program, when executed by a processor, implements the steps of the method of claim 1.
